(19) United States
(12) Patent Application Publication
(10) Pub. No.: US 2025/0375496 A1
(43) Pub. Date: Dec. 11, 2025

(54) SPECIES SPECIFIC PROTEINS FOR WOUND HEALING AND METHODS OF USE

(71) Applicant: EIR Pharmaceuticals, LLC, Olathe, KS (US)

(72) Inventors: William P Duncan, Olathe, KS (US); Lauren S Gollahon, Lubbock (YS); D. Andrew Skaff, Kansas City, MO (US)

(21) Appl. No.: 19/240,787

(22) Filed: Jun. 17, 2025

Related U.S. Application Data
(63) Continuation-in-part of application No. 18/115,929, filed on Mar. 1, 2023, now Pat. No. 12,357,670.
Application No. 18/115,929 is a continuation of application No. 16/686,535, filed on Nov. 18, 2019, now Pat. No. 11,607,438.
Application No. 16/686,535 is a continuation-in-part of application No. 16/590,807, filed on Oct. 2, 2019, now Pat. No. 11,622,987.
(60) Provisional application No. 62/740,047, filed on Oct. 2, 2018.
(60) Provisional application No. 62/788,261, filed on Jan. 4, 2019.
(60) Provisional application No. 63/660,832, filed on Jun. 17, 2024.

(51) Int. Cl. A61K 36/886; A61K 8/19; A61K 8/34; A61K 8/37; A61K 8/64; A61K 8/97; A61K 8/98; A61K 9/06; A61K 31/198; A61K 47/02; A61K 47/10; A61K 47/44; A61P 17/02; A61Q 19/00

(52) U.S. Cl. CPC A61K 36/886; A61K 8/19; A61K 8/342; A61K 8/345; A61K 8/347; A61K 8/375; A61K 8/64; A61K 8/97; A61K 8/987; A61K 9/06; A61K 31/198; A61K 47/02; A61K 47/10; A61K 47/44; A61P 17/02; A61Q 19/00; A61K 2236/30; A61K 2800/592

(57) ABSTRACT

TRIM72, is a naturally occurring protein in mammalian cells that is known to be important in cell membrane repair and tissue regeneration in injury conditions. When tissues are damaged, (abrasions, cuts, scrapes, etc.), the protein is recruited to the damaged tissue to aid in reassembly of the lipid membrane layer through shuttling membrane vesicles. The more quickly the cell membrane can be repaired, possibly even saving the damaged cells, the faster the full healing process will occur. This also greatly reduces the risk of infection by minimizing the length of time the damaged cells are exposed to the outside world. Rather than waiting for the protein to be naturally produced and recruited to the injury site, a topical application of the purified protein, either directly or contained as an active ingredient within a hydrogel or cream, could be applied, delivering the protein in high quantities directly to the affected area.

### CROSS-REFERENCE TO RELATED APPLICATION

This application is a continuation-in-part of and claims priority in U.S. patent application Ser. No. 18/115,929 filed Mar. 1, 2023, which is a continuation of and claims priority in U.S. patent application Ser. No. 16/686,535 filed Nov. 18, 2019, now U.S. Pat. No. 11,607,438 Issued Mar. 1, 2023, which is a continuation-in-part of and claims priority in U.S. patent application Ser. No. 16/590,807 filed Oct. 2, 2019, now U.S. Pat. No. 11,622,987 Issued Mar. 22, 2023 which claims priority in U.S. Provisional Patent Application Nos. 62/740,047 filed Oct. 2, 2018 and 62/788,261 filed Jan. 4, 2019, and this application also claims priority in U.S. Provisional Patent Application No. 63/660,832 filed Jun. 17, 2024, all of which are incorporated herein by reference.

### BACKGROUND OF THE INVENTION

### 1. Field of the Invention

The present invention relates generally to a series of recombinant proteins associated with wound healing properties, and specifically a member of the TRIM family of proteins, MG-53, that has been derived from a series of animal sources.

### 2. Description of the Related Art

Existing methods for generating recombinant proteins and treating wounds do exist. However, existing wound care methods relate to anti-microbials and/or providing pain relief rather than adequately and reliably assisting directly in wound healing. The recombinant human MG-53 protein has been generated for wound healing, however, animal sources may be more appropriate for use for specific purposes.

Heretofore there has not been an available method for the preparation of these proteins with the advantages and features of the present invention.

### BRIEF SUMMARY OF THE INVENTION

The present invention generally provides for the preparation of a recombinant protein from the TRIM family, MG-53, that is associated with wound healing properties in organisms in the animal kingdom, and use of said composition for the treatment of wounds internally or externally. The resulting composition may then be combined into a topical cream or other solution for use.

### BRIEF DESCRIPTION OF THE DRAWINGS

The drawings constitute a part of this specification and include exemplary embodiments of the present invention illustrating various objects and features thereof.

FIG. 1 is a diagram of the phylogenetic comparison of the sequences referenced in Table 1. Sequences for animals that are relatively similar group closely as compared to the similarity of the animals themselves.

FIG. 2 is a diagram of a structural alignment of the protein sequences referenced in Table 1 and FIG. 1. Each animal protein sequence is threaded through the human MG-53 protein structure (Protein Data Bank structure ID: 3KB5). Residues that are not conserved between the sequences are shown as sticks.

FIG. 3 is a diagram depicting a time-course of a wound healing after application of an effective amount of purified, recombinant human MG-53 protein in a topical hydrogel application.

FIG. 4 is a diagram of the amplification of the MG-53 gene from endogenous bovine mRNA followed by expression of recombinant bovine MG-53 protein in a bacterial expression system.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed manifestation.

Certain terminology will be used in the following description for convenience in reference only and will not be limiting. For example, up, down, front, back, right and left refer to the invention as orientated in the view being referred to which the referral is directed. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment Method for Generating MG-53 Recombinant Proteins and Purification Process

DNA sequences for companion animals; dog and cat; livestock animal; horse, cow, and pig; and exotic animals; camel, tiger, frog and cobra; were downloaded from the NIH GenBank public database. Each DNA sequence was optimized for codon usage for a bacterial, yeast or mammalian cellular expression system prior to synthesis of the plasmid. An expression plasmid for each sequence was generated and engineered to impart a specific antibiotic resistance to ensure resulting cell cultures are unadulterated, and a N- or C-terminal affinity tag for purification of the resulting recombinant protein via the purification method. Each plasmid is then transformed into an appropriate expression vector. Cell cultures are raised prior to cell collection via centrifugation. Cells are lysed and the aqueous phase is passed over an affinity matrix to isolate the recombinant protein. The resulting partially purified protein may be further purified by additional methods as required to reach the desired purity level.

The use of the recombinant protein in a topical preparation for treatment of damage to the skin, such as: cuts, scrapes, abrasions, gouges, burns, or other external wounds.

A second use of the recombinant protein is internally following surgical or non-surgical procedures.

A third use of the recombinant protein is as a treatment for organ injuries or recovery from surgical replacement.

A fourth use of the recombinant protein is as a treatment for respiratory disorders.

A fifth use of the recombinant protein is as a prophylactic increasing the growth protection of cardiovascular function from ischemia.

A sixth use of the recombinant protein is as a topical treatment to reduce the potential of scarring and reduction of existing scarring.

A seventh use of the recombinant protein is as a topical cosmetic application.

An eighth use of the recombinant protein is for prophylactic protection of respiratory function against chemically-induced damage and respiratory infections.

A ninth use of the recombinant protein is in treatment of digestive disorders of the stomach, small and large intestines.

A tenth use of the recombinant protein in treatment of liver disorders.

It is to be understood that while certain embodiments and/or aspects of the invention have been shown and described, the invention is not limited therein and encompasses various other embodiments and aspects.

### III. Examples and Results Utilizing the Resultant Product of Purification of Recombinant Species-Specific MG-53 Proteins

The human TRIM72/MG-53 DNA sequence was searched for relatively close matches in the NIH GenBank database. Two different equine genes (E. caballas and E. przewalskii) were located and downloaded, along with genes from dog (C. lupus familiaris), cat (F. catus), cow (B. taurus), and pig (S. scrofa), which are sources for possible animal health applications, then also camel (C. dromedarius), frog (X. tropicalis), King cobra (O. hannah) and Siberian tiger (P. tigris altaica), for additional diversity. These sequences were aligned as shown in Table 1 and compared to ensure that the annotated genes from the database are correct and are likely to be the correct target. Table 1 is a multiple sequence alignment of the MG-53 proteins from B. taurus, S. scrofa, P. tigris altaiaca, F. catus, E. caballus, E. przewalskii, C. lupus familiaris, X. tropicalis and O. hannah with H. sapiens depicted for comparative purposes. Each animal protein shows similarity to the human sequence, but each is less than 95% identical as compared to the human sequence reference.

TABLE 1

| | |
|---|---|
| Felis catus | ....MSAAPG LLHQELSCPL CLQLFDAPVT AECGHSFCRA CLSRVAG.EP |
| Panthera tigris | ....MSAAPG LLHQELSCPL CLQLFDAPVT AECGHSFCRA CLSRVAG.EP |
| Canis lupus | ....MSAAPG LLHQELSCPL CLQLFDAPVT AECGHSFCRA CLSRVAG.EP |
| Equus caballus | ....MSAAPG LLHQELSCPL CLQLFDAPVT AECGHSFCRA CLSRVAG.EP |
| Equus | ....MSAAPG LLHQELSCPL CLQLFDAPVT AECGHSFCRA CLSRVAG.EP |
| przewalskii | |
| Sus scrofa | ....MSAAPG LLHQELSCPL CLQLFDAPVT AECGHSFCRA CLGRVAG.EP |
| Camelus | ....MSAAPG LLHQELSCPL CLQLFDAPVT AECGHSFCRA CLNRVAG.EP |
| dromedarius | |
| Bos taurus | ....MSAAPG LLHQELSCPL CLQLFDAPVT AECGHSFCRA CLSRVAG.EP |
| Homo sapiens | ....MSAAPG LLHQELSCPL CLQLFDAPVT AECGHSFCRA CLGRVAG.EP |
| Xenopus | ..MSTPQLMQ GMQKDLTCPL CLELFRAPVT PECGHTFCQG CLTGAPKNQD |
| tropicalis | |
| Ophiophagus | MSSSQQRLMQ GMHQDLSCPI CLKLFQSPVT TECGHTFCQA CLARVATEES |
| hannah | |
| Felis catus | AADGTVPCPC CQALTRPQAL NTNLQLARLV EGLAQVPQGH CEEHLDPLSI |
| Panthera tigris | AADGTVPCPC CQALTRPQAL STNLQLARLV EGLAQVPQGH CEEHLDPLSI |
| Canis lupus | AADGTVPCPC CQALTRPQAL STNQQLARLV EGLAQVPQGH CEEHLDPLSI |
| Equus caballus | AADGTVLCPC CQAPTRPQAL STNQQLARLV EGLAQVPQGH CEEHLDPLSI |
| Equus | AADGTVLCPC CQAPTRPQAL STNQQLARLV EGLAQVPQGH CEEHLDPLSI |
| przewalskii | |
| Sus scrofa | AADGTVLCPS CQAPTRPQAL STNQQLARLV EGLAQVPQGH CEEHLDPLSI |
| Camelus | AADGTVLCPS CQAPTRPQAL STNQQLARLV EGLAQVPQGH CEEHLDPLSI |
| dromedarius | |
| Bos taurus | AADGTVLCPS CQAPTRPQAL STNLQLARLV EGLAQVPQGH CEEHLDPLSI |
| Homo sapiens | AADGTVLCPC CQAPTRPQAL STNLQLARLV EGLAQVPQGH CEEHLDPLSI |
| Xenopus | QNGSTP.CPT CQTPSRPETL QINRQLEHLV QSFKQVPKGH CLEHLDPLSV |
| tropicalis | |
| Ophiophagus | EGKTTPSCPT CQALTKVEQL RINQQMEHLV QSFRQVPRDH CEEHLDPLSV |
| hannah | |
| Felis catus | YCEQDRALVC GVCASLGSHR GHRLLPAAEA HARLKTQLPQ QKMQLQEACM |
| Panthera tigris | YCEQDRALVC GVCASLGSHR GHRLLPAAEA HARLKTQLPQ QKMQLQEACM |
| Canis lupus | YCEQDRALVC GVCASLGSHR GHRLLPAAEA HARLKTQLPQ QKLQLQGACM |
| Equus caballus | YCEQDRALVC GVCASLGSHR GHRLLPAAEA HARLKTQLPQ QKMQLQEACM |
| Equus | YCEQDRALVC GVCASLGSHR GHRLLPAAEA HARLKTQLPQ QKMQLQEACM |
| przewalskii | |
| Sus scrofa | YCEQDRVLVC GVCASLGSHR GHRLLPAAEA HARLKTQLPQ QKLQLQEACM |
| Camelus | YCEQDRALVC GVCASLGSHR GHRLLPAAEA HARLKTQLPQ QKLQLQEACM |
| dromedarius | |
| Bos taurus | YCEQDRALVC GVCASLGSHR GHRLLPAAEA HARLKTQLPQ QKMQLQEACM |
| Homo sapiens | YCEQDRALVC GVCASLGSHR GHRLLPAAEA HARLKTQLPQ QKLQLQEACM |
| Xenopus | YCEQDKELIC GVCASLGKHK GHNIITAAEA YAKLKRQLPQ QQVILQEARL |
| tropicalis | |
| Ophiophagus | YCEQDRQVIC GVCASLGKHK GHNIITAAEA HQRMKKQLPQ QQVQLQEAQI |
| hannah | |
| Felis catus | RKEKSVAVLE HQLLEVEETV R........Q FRGAVGEQLG KMRVFLAALE |
| Panthera tigris | RKEKSVAVLE HQLLEVEETV R........Q FRGAVGEQLG KMRVFLAALE |
| Canis lupus | RKEKSVALLE HQLMEVEEMV R........Q FRGAVGEQLG KMRVFLAALE |
| Equus caballus | RKEKSVAVLE HQLVEVEEMV R........Q FRGAVGEQLG KMRVFLAALE |
| Equus | RKEKSVAVLE HQLVEVEEMV R........Q FRGAVGEQLG KMRVFLAALE |
| przewalskii | |
| Sus scrofa | RKEKSVGVLE QQLVEVEETV R........Q FRGAVGEQLG KMRLFLAALE |
| Camelus | RKEKSVAVLE HQLAEVEETV R........Q FRGAVGEQLG KMRLFLAALE |
| dromedarius | |
| Bos taurus | RKEKSVALLE HQLLEVEETV R........Q FRGAVGEQLG KMRLFLAALE |
| Homo sapiens | RKEKSVAVLE HQLVEVEETV R........Q FRGAVGEQLG KMRVFLAALE |
| Xenopus | KKEKTVAVLD RQVAEVQD.. ......TVSR FKGNVKHQLN AMRSYLSIME |
| tropicalis | |
| Ophiophagus | RKEKTIALLN RQIAEVEVSV LDVCLSLLSR FKHQVAEQLG VMRSYLGVLE |
| hannah | |
| Felis catus | GSLDREAERV RGEAGVALRR ELGSLNSYLE QLRQMEKVLE EVADKPQTEF |
| Panthera tigris | GSLDREAERV RGEAGVALRR ELGSLNSYLE QLRQMEKVLE EVADKPQTEF |
| Canis lupus | GSLDREAERV RGGAGVALRR ELGSLNSYLE QLRQMEKVLE EVADKPQTEF |
| Equus caballus | GSLDREAERV RGEAGVALRR ELGSLNSYLE QLRQMEKVLE EVADKPQTEF |
| Equus | GSLDREAERV RGEAGVALRR ELGSLNSYLE QLRQMEKVLE EVADKPQTEF |
| przewalskii | |
| Sus scrofa | GSLDREAERV RGEAGVALRR ELGSLKSYLE QLRQMEKVLE EVADKPQTEF |
| Camelus | GSLDREAERV RGEAGVALRR ELGGLNSYLE QLRQMEKVLE EVADKPQTEF |
| dromedarius | |
| Bos taurus | GSLDREAERV RGEAGVALRR ELGSLNSYLE QLRQMEKVLE EVADKPQTEF |
| Homo sapiens | GSLDREAERV RGEAGVALRR ELGSLNSYLE QLRQMEKVLE EVADKPQTEF |
| Xenopus | ASLSKEADNA EHTATEALLV ERKTMGHYLD QLRQMDGVLK DVESQEQTEF |
| tropicalis | |
| Ophiophagus | SSLGQEAEKV QQQASEALQN ERKTVTRYLD QLKQMEMVLG EVQDEPQTEF |
| hannah | |
| Felis catus | LMKYCLVTSR LQKILAESPP PARLDIQLPV ISDDFKFQVW RKMFRALMPA |
| Panthera tigris | LMKYCLVTSR LQKILAESPP PARLDIQLPV ISDDFKFQVW RKMFRALMPA |
| Canis lupus | LMKYCLVTSR LQKILAESPP PARLDIQLPV ISDDFKFQVW RKMFRALMPV |
| Equus caballus | LMKYCLVTSR LQKILGESPP PARLDIQLPV ISDDFKFQVW RKMFRALMPA |
| Equus | LMKYCLVTSR LQKILGESPP PARLDIQLPV ISDDFKFQVW RKMFRALMPA |
| przewalskii | |
| Sus scrofa | LMKYCLVTSR LQKILAESPP PARLDIQLPV ISDDFKFQVW RKMFRALMPA |
| Camelus | LMKYCLVTSR LQKILAESPP PARLDIQLPV ISDDFKFQVW RKMFRALMPA |
| dromedarius | |
| Bos taurus | LMKYCLVTSR LQKILAESPP PARLDIQLPI ISDDFKFQVW RKMFRALMPA |
| Homo sapiens | LMKYCLVTSR LQKILAESPP PARLDIQLPI ISDDFKFQVW RKMFRALMPA |
| Xenopus | LRKYCVVAAR LNKILAESPP PGRLDIQLPI ISDEFKFQVW RKMFRALMPA |
| tropicalis | |
| Ophiophagus | LRKYCLVASR LQKILGESPP IARMDIQLPI ISDEFKFQVW RKMFRAIMPA |
| hannah | |
| Felis catus | LMKYCLVTSR LQKILAESPP PARLDIQLPV ISDDFKFQVW RKMFRALMPA |
| Panthera tigris | LMKYCLVTSR LQKILAESPP PARLDIQLPV ISDDFKFQVW RKMFRALMPA |
| Canis lupus | LMKYCLVTSR LQKILAESPP PARLDIQLPV ISDDFKFQVW RKMFRALMPV |
| Equus caballus | LMKYCLVTSR LQKILGESPP PARLDIQLPV ISDDFKFQVW RKMFRALMPA |
| Equus | LMKYCLVTSR LQKILGESPP PARLDIQLPV ISDDFKFQVW RKMFRALMPA |
| przewalskii | |
| Sus scrofa | LMKYCLVTSR LQKILAESPP PARLDIQLPV ISDDFKFQVW RKMFRALMPA |
| Camelus | LMKYCLVTSR LQKILAESPP PARLDIQLPV ISDDFKFQVW RKMFRALMPA |
| dromedarius | |
| Bos taurus | LMKYCLVTSR LQKILAESPP PARLDIQLPI ISDDFKFQVW RKMFRALMPA |
| Homo sapiens | LMKYCLVTSR LQKILAESPP PARLDIQLPI ISDDFKFQVW RKMFRALMPA |
| Xenopus | LRKYCVVAAR LNKILAESPP PGRLDIQLPI ISDEFKFQVW RKMFRALMPA |
| tropicalis | |
| Ophiophagus | LRKYCLVASR LQKILGESPP IARMDIQLPI ISDEFKFQVW RKMFRAIMPA |
| hannah | |
| Felis catus | MKELTFDPST AHPSLVVSPS GRRVECLDQK APPAGEDPCQ FDKAVAVVAH |
| Panthera tigris | MKELTFDPST AHPSLVVSPS GRRVECLDQK APLAGEDPCQ FDKAVAVVAH |
| Canis lupus | TKELTFDPSS AHPSLVLSPS GRRVECSDQK APPAGEDPCQ FDKAVAVVAQ |
| Equus caballus | LKELTFDPCS AHPSLVLSPS GRRVECSEQK APPAGEDPCQ FDKAVAVVAH |
| Equus | LKELTFDPCS AHPSLVLSPS GRRVECSEQK APPAGEDPCQ FDKAVAVVAH |
| przewalskii | |
| Sus scrofa | MQELTFDPST AHPSLVLSAS GRRVECSEQK APPAGEDPRQ FDKAVAVVTH |
| Camelus | LQELTFDPST AHPSLVLSAS GRRVECSEQK PPPAGDDPCQ FDKAVAVVTH |
| dromedarius | |
| Bos taurus | RQELTFDPST AHPSLVLSNS GRCVECSEQK APPAGEDPRQ FDKAVAVVTH |
| Homo sapiens | LEELTFDPSS AHPSLVVSSS GRRVECSEQK APPAGEDPRQ FDKAVAVVAH |
| Xenopus | LENLTFDPDT AQQNLVVFSD GKSVECSEQK QSVS.DEPNR FDKSNCLVSK |
| tropicalis | |
| Ophiophagus | LENLTEDPAT AQANLVVSED GKRVECVEHK QAVSTDDPQR FDKSNCLVSS |
| hannah | |
| Felis catus | QLLSDGEHYW EVEVGDKPRW ALGVILAQAS RRGRLHAVPS QGLWLLGLRE |
| Panthera tigris | QLLSDGEHYW EVEVGDKPRW ALGVISAQAS RRGRLHAVPS QGLWLLGLRE |
| Canis lupus | QVLSDGEHYW EVQVGEKPRW ALGVIAAQAS RRGRLHAVPS QGLWLLGLRD |
| Equus caballus | QLLSDGEHYW EVEVGDKPRW ALGVIAAQAN RRGRLHAVPS QGLWLLGLRD |
| Equus | QLLSDGEHYW EVEVGDKPRW ALGVIAAQAN RRGRLHAVPS QGLWLLGLRD |
| przewalskii | |
| Sus scrofa | QLLSEGEHYW EVEVGDKPRW ALGVIGAQAG RRGRLHAVPS QGLWLLGLRE |
| Camelus | QLLSEGEHYW EVEVGDKPRW ALGVISAQAG RRGRLHAVPS QGFWLLGLRD |
| dromedarius | |
| Bos taurus | QLLSEGEHYW EVEVGDKPRW ALGVIGAQAG RRGRLHAVPS QGLWLLGLRD |
| Homo sapiens | QQLSEGEHYW EVDVGDKPRW ALGVIAAEAP RRGRLHAVPS QGLWLLGLRE |
| Xenopus | ESFTEGEHYW EVLVEDKPRW ALGVISETAN RKGKLHASPS NGFWLIGCKE |
| tropicalis | |
| Ophiophagus | QSFSQGEHYW EVTVDDKPRW ALGVISAETG RKGRLHATPS NGFWLVGCKE |
| hannah | |
| Felis catus | GKILEAHVEA KEPRALRTPE RRPLRIGIYL SFGDGILSFY DASNADALEQ |
| Panthera tigris | GKILEAHVEA KEPRALRTPE RRPLRIGIYL SFGDGILSFY DASNADALEQ |
| Canis lupus | GKILEAHVEA KEPRALRTPE RRPTRIGIYL SFGDGVLSFY DASDPDALEL |
| Equus caballus | GKILEAHVEA KEPRALRTPE RRPTRIGIYL SFGDGVLSFY DASDPDALEL |
| Equus | GKILEAHVEA KEPRPLRTPE RRPSRIGIYL SFGDGVLSFY DASDADALEL |
| przewalskii | |
| Sus scrofa | GKILEAHVEA KEPRALRTPE RRPSRIGIYL SFADGVLSFY DASDADALEL |
| Camelus | AKILEAHVEA KEPRALRTPE RRPTRIGIYL SFGDGVLSFY DASDADALEL |
| dromedarius | |
| Bos taurus | GKILEAHVEA KEPRALRTPE RRPTRIGIYL SFGDGVLSFY DASDPDALEL |
| Homo sapiens | GKILEAHVEA KEPRALRSPE RRPTRIGLYL SFGDGVLSFY DASDADALVP |
| Xenopus | GKVYEAHTEQ KEPRVLR.VE GRPEKIGIYL SFSDGVVSFF DSSDEDNIKL |
| tropicalis | |
| Ophiophagus | GKSYEAYVEH KEPRSLK.LE GKPSRIGIYL SEDDGLLAFY DASDEDNLVQ |
| hannah | |
| Felis catus | LFAFHERLPG PVYPFFDVCW HDKGKNAQPL LLVGPDGEEA ..... |
| Panthera tigris | LFAFHERLPG PVYPFFDVCW HDKGKNAQPL LLVGPDGEEA ..... |
| Canis lupus | LFAFHERLPG PVYPFFDVCW HDKGKNAQPL LLVGPDGEEA ..... |
| Equus caballus | LFAFHERLPG PVYPFFDVCW HDKGKNTQPL LLVGPDGEEA ..... |
| Equus | LFAFHERLPG PVYPFFDVCW HDKGKNTQPL LLVGPDGEEA ..... |
| przewalskii | |
| Sus scrofa | LFAFHERLPG PVYPFFDVCW HDKGKNAQPL LLVGPDSGGE A.... |
| Camelus | LFAFHERLPG PVYPFFDVCW HDKGKNAQPL LLVGPDSGGE A.... |
| dromedarius | |
| Bos taurus | LFAFHERLPG PVYPFFDVCW HDKGKNAQPL LLVGPEVSSG SGSEA |
| Homo sapiens | LFAFHERLPR PVYPFFDVCW HDKGKNAQPL LLVGPEGAEA ..... |
| Xenopus | LYTFNERFSG RLHPFFDVCW HDKGKNAQPL KIFYPPAEQL ..... |
| tropicalis | |
| Ophiophagus | IFAFRERFTG TAYPFFDVCW HDKGKNSQPL IIYTPESQER ..... |
| hannah | |

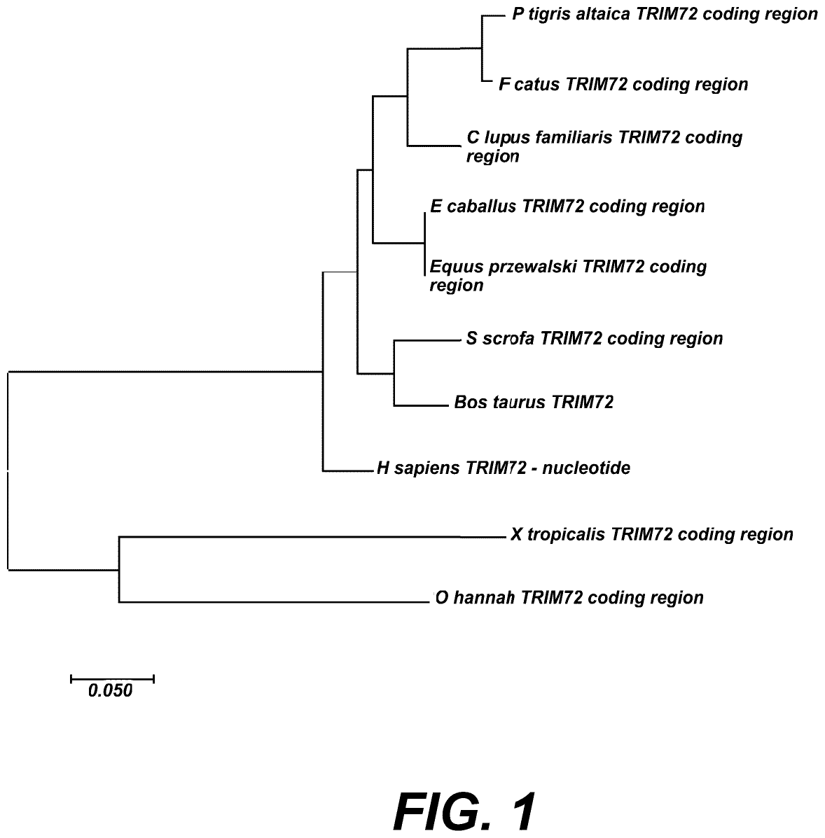

A phylogenetic analysis was performed as depicted in FIG. 1. As expected, the human gene is relatively similar to pig (S. scrofa); the housecat and tiger genes are closely related; the two horse genes couple closely together; and frog and cobra demonstrating the most divergence from the human gene. This data combined matches closely with expectation for typical genetic variation. The horse gene is very likely correctly annotated, and the sequence is similar, but not identical to the human version.

The DNA sequence sets were translated to their corresponding amino acid sequences for the more practical comparison. The results of the analysis are compiled in Table 2. The sequence similarity between human and the various species is extremely high. Even with the evolutionary distance between frog/cobra to human, the proteins are still 84% homologous. It is a little surprising that there is more variation in the SPRY domain (which makes up nearly half of the full-length protein and has the more defined structure) as compared to the opposite half of the protein, which is mostly an extended α-helix, which would be almost entirely solvent-exposed surface.

TABLE 2
Protein sequence similarity of TRIM72 between human and various species

| | H. sapiens TRIM72 | H. sapiens TRIM72 |
|---|---|---|
| | whole protein:whole protein | SPRY domain:SPRY domain |
| | (% identity/% homology) | (% identity/% homology) |
| F. catus | 93.7/97.3 | 89.1/94.8 |
| C. lupis familiaris | 93.5/97.1 | 89.1/95.3 |
| B. taurus | 94.7/98.1 | 90.2/95.9 |
| S. scrofa | 93.9/98.3 | 90.7/97.4 |
| E. caballas | 94.2/97.6 | 88.6/95.1 |
| E. przewalskii | 94.3/97.5 | 90.2/95.3 |
| C. dromedarius | 93.5/97.9 | 89.1/96.4 |
| P. tigris altaica | 92.0/96.8 | 88.6/94.8 |
| X. tropicalis | 59.1/84.1 | 62.1/84.7 |
| O. hannah | 61.5/83.5 | 64.1/85.9 |

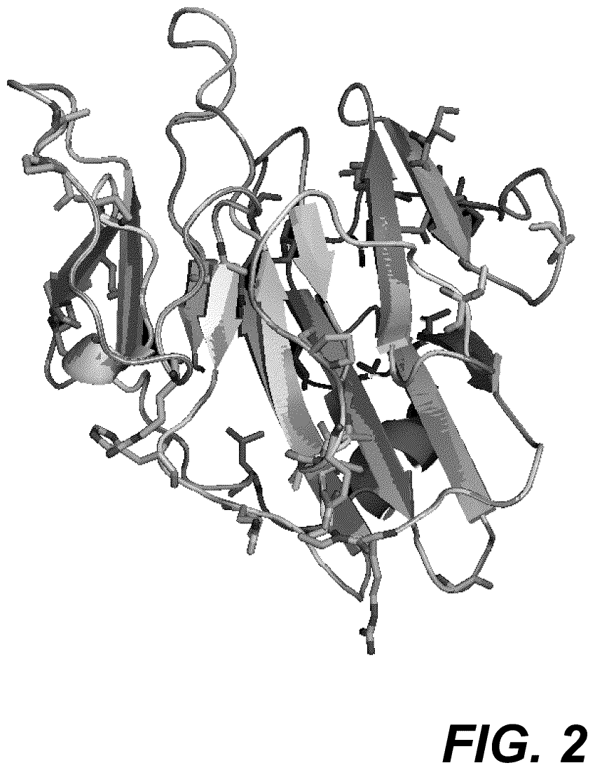

A structural alignment for all 11 TRIM72/MG-53 proteins modeled/aligned to the human structure from Protein Data Bank (3KB5) as shown in FIG. 2. Residues that are not conserved are shown as sticks in FIG. 2. Nearly all the non-conserved residues are located on the surface of the structural models. Because of this, it is likely that the non-human versions of the protein are likely to have the same function and similar relative activities.

### IV. Additional Examples

Recent work included in patent #U.S. Pat. No. 11,607,438B2, demonstrating this process. Purified, recombinant human TRIM72/MG-53 was included as the active ingredient in a topical hydrogel base and applied to wounds in both controlled and uncontrolled environments with great success. In the laboratory, the topical mixture was applied to mice with induced injury and showed signs of healing at only 1 day post initial treatment. Outside of the laboratory, the topical application has also demonstrated a rapid healing time with little to no scarring in serious injuries sustained by horses.

The product used in these experiments was the human TRIM72/MG-53 protein expressed in E. coli, purified, and then formulated in a hydrogel for use.

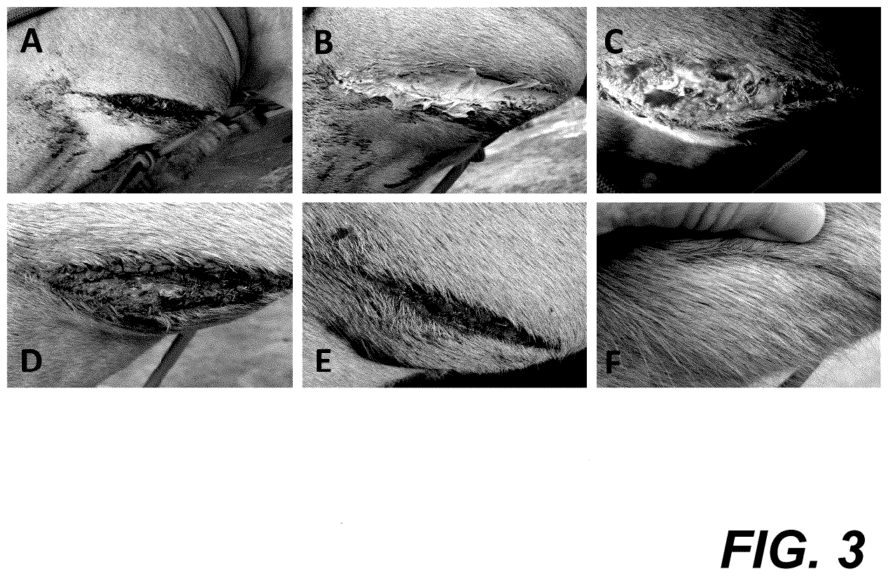

FIG. 3 is an example of wound treatment containing human MG-53 as the active ingredient in an emulsifier system applied topically. Panel A shows a cheek laceration of an eleven-year-old quarter horse mare. This would normally be sutured, instead the wound was left open to study the effectiveness of the cream to heal said wound. The wound was not washed or disinfected prior to addition of the cream. The wound was treated with the hydrogel containing the human recombinant TRIM72/MG53 protein as an active ingredient, twice per day and was not washed or otherwise treated for the duration of healing. Panel B shows the application of the cream to the wound. The cream remained where it was applied and did not cause any irritation to the wound or surrounding areas. Panel C shows the wound at Day 4 where indications of the wound repair can be seen. Panel D shows the wound at Day 8. No serum drainage had been detected from the wound and appeared to undergo less debridement in the repair process than is typically seen. Panel E shows the wound on Day 15 where the wound has constricted considerably. Panel F shows the wound on Day 58 where the wound has been repaired and hair has regrown with its original pigmentation, indicating normal, functional dermal cell regeneration. The scar line is only barely visible.

Rather than using a genetic mismatch for the intended use in horses, it is more appropriate to use the equine version of the protein. The current product demonstrated in the patent is a topical application that delivers TRIM72, a protein that the organism already produces (in this case—a mismatch, human version of the same protein the horse would be producing), to an open wound as directly as possible.

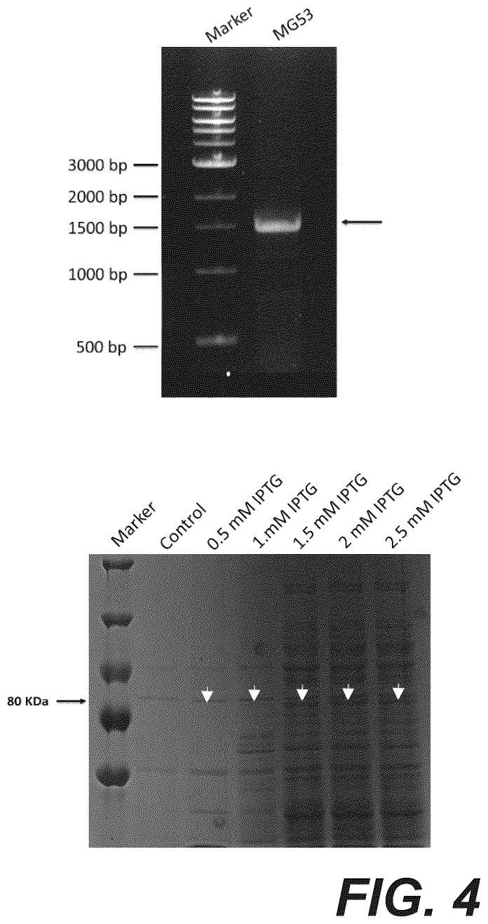

FIGS. 4A and 4B show the results of the key steps in cloning the MG-53 gene from a bovine source. Total RNA was extracted using a kit method, ensuring RNA integrity via gel electrophoresis and quantification via a Nanodrop. Complementary DNA (cDNA) was synthesized using a reverse transcription kit, serving as a template for downstream applications. Gene-specific primers were designed to include restriction enzyme sites of KpnI and XhoI for cloning of MG-53 into the pEGFP-C1 vector. Amplification of the MG-53 gene was performed under optimized conditions, and the product was visualized using a 1% agarose gel. The 1.5 kb MG-53 gene was amplified with high fidelity, as shown in Lane 2 of FIG. 4A.

The amplified MG-53 gene and pEGFP-C1 vector were digested with restriction enzymes (e.g., KpnI and XhoI) and then ligated. The ligated products were transformed into E. coli competent cells (DH5α) using kanamycin as a selectable marker. Positive clones were screened and confirmed by colony PCR and restriction digestion and further confirmed by sequencing.

The pEGFP-C1+BvMG53 construct was transformed into bacterial expression vector BL21 to induce the expression of MG-53 at 18° C. and at different IPTG (0.5 mM, 1 mM, 1.5 mM, 2 mM and 2.5 mM) concentrations to optimize the conditions (e.g., temperature, IPTG concentration) for MG-53 expression as shown in FIG. 4B. The MG-53 protein fused with an EGFP tag showed expression at all IPTG concentrations, but the expression is higher at 1.5 mM, 2 mM, and 2.5 mM IPTG induction. The molecular weight of bovine MG-53 protein is 54 kDa, and EGFP is 26 kDa. Because MG-53 is tagged with EGFP so the total weight of EGFP+BvMG53 is 80 kDa as shown in FIG. 4B.

Having thus described the invention, what is claimed as new and desired to be secured by Letters Patent is:

1. A preparation for treatment purposes, the preparation comprising:
a DNA sequences for MG53e generated from samples of the species including: B. taurus, S. scrofa, P. tigris altaiaca, F. catus, E. caballus, E. przewalskii, C. dromedarius, C. lupus familiaris, X. tropicalis and O. hannah;
each of said samples being individually optimized for codon usage for expression in a first cell culture;
an expression plasmid configured to impart antibiotic resistance configured to result in unadulterated cell cultures;
said plasmid configured to be transformed into an expression vector;
a raised second cell culture from said expression vector, said second cell culture selected from the list of cell cultures comprising: bacterial, yeast, and mammalian;
collected cells of said raised cultures configured to be lysed, whereafter an aqueous phase is passed over an affinity matrix configured to isolate a recombinant protein;
said recumbent protein comprising a C-terminal affinity tag configured for purification of said recumbent protein; and
whereby said recombinant protein is purified to at least seventy percent (70%) purity.

2. The preparation of claim 1, wherein said preparation comprises a topical application configured to treat damaged skin.

3. The preparation of claim 1, further comprising:
said preparation comprises an internal preparation, said internal preparation including said recombinant protein;
wherein said preparation is configured to treat internal organs; and
wherein said preparation configured to be delivered via a delivery method selected from the list comprising: intravenously; and orally.

4. The preparation of claim 1, further comprising:
said preparation prepared into a delivery method selected from the list comprising: vaporized; and aerosolized;
said preparation configured to be inhaled such as to coat lung tissue linings; and
wherein said preparation is configured to treat respiratory disorders.

5. The preparation of claim 1, further comprising:
said preparation being topically applied; and
whereby said preparation is configured for cosmetic purposes.

6. The preparation of claim 1, further comprising:
said preparation comprises an internal preparation, said internal preparation including said recombinant protein; and
whereby said preparation is configured for use as a prophylactic increasing the growth protection of cardiovascular function from ischemia.

7. The preparation of claim 1, further comprising:
said preparation being topically applied; and
whereby said preparation is configured for prevention or reduction of scarring.

8. The preparation of claim 1, further comprising:
said preparation prepared into a delivery method selected from the list comprising: vaporized; and aerosolized;
said preparation configured to be inhaled such as to coat lung tissue linings; and
whereby said preparation is configured for prophylactic protection of respiratory function against chemically-induced damage and respiratory infections.

9. The preparation of claim 1, further comprising:
said preparation comprises an internal preparation, said internal preparation including said recombinant protein;
wherein said preparation is configured to treat digestive disorders of the stomach, small intestine, and large intestine; and
wherein said preparation configured to be delivered via a delivery method selected from the list comprising: intravenously; and orally.

10. The preparation of claim 1, further comprising:
said preparation comprises an internal preparation, said internal preparation including said recombinant protein;
wherein said preparation is configured whereby said preparation is configured for the treatment of liver disorders; and
wherein said preparation configured to be delivered via a delivery method selected from the list comprising: intravenously; and orally.
